(19) United States
(12) Patent Application Publication
(10) Pub. No.: US 2026/0215979 A1
(43) Pub. Date: Jul. 30, 2026

(54) BUILDING STRUCTURES THAT FUNCTION AS A MEDICAL FACILITY

(71) Applicant: Renasolve, Inc., Cheyenne, WY (US)

(72) Inventor: Rubin Chandran, Cheyenne, WY (US)

(73) Assignee: Renasolve, Inc., Cheyenne, WY (US)

(21) Appl. No.: 19/464,427

(22) Filed: Jan. 29, 2026

Related U.S. Application Data
(60) Provisional application No. 63/751,086, filed on Jan. 29, 2025.

(51) Int. Cl. A61G 3/00; A61G 10/00; A61G 12/00; A61M 1/14

(52) U.S. Cl. CPC A61G 3/003; A61G 10/005; A61G 12/002; A61M 1/14; A61G 2220/00; A61M 2205/84

(57) ABSTRACT

A structure for use as a medical facility includes a plurality of discrete structural units placed side-by-side relative to each other. At least one of the structural units has at least one patient station that includes a patient chair and a dialysis machine.

### CROSS REFERENCE TO RELATED APPLICATIONS

This application claims the benefit of U.S. Provisional Patent Application No. 63/751,086, filed Jan. 29, 2025, which is incorporated herein by reference.

### FIELD

The present application relates to building structures that function as a medical facility, and in particular, modular building structures used for treating patients requiring dialysis.

### BACKGROUND

Rural areas often lack medical facilities that can treat patients requiring dialysis. Oftentimes, patients must travel relatively far distances to the nearest treatment center, typically three times a week. In some cases, patients may need to relocate to a larger urban area to access dialysis treatment.

### SUMMARY

The present application relates to building structures that can function as a medical facility, and in particular, a medical facility for performing dialysis on patients and/or for examining or otherwise treating dialysis patients. The building structures can comprise one or more individual or discrete structural units, such as in the form of standard shipping containers, which can be manufactured at a manufacturing site and transported to a geographic area via traditional transportation methods, such as by tractor trailer, train, or by maritime transport on a vessel. The individual structural units can be assembled at a desired geographic location to form a fully functioning medical facility for serving patients in the area. Some rural areas may lack adequate conventional brick and mortar structures that can be used as a dialysis center and/or may lack the ability to construct a new facility for serving the local population in a cost-efficient manner. Thus, the building structures disclosed herein can be used in geographic areas in need of dialysis centers. In some examples, the manufacture, transport and assembly of the structural units can be accomplished more efficiently and at a lower cost than the construction of a conventional brick and mortar medical facility.

In some examples, the building structures disclosed herein can be referred to as modular building structures, as they are assembled from a plurality of similarly or identically sized structural units. The number of structural units needed to form a building structure that functions as a medical facility can be selected to meet the needs of the medical facility.

In some examples, some or all of the required medical equipment (such as dialysis machines) and other equipment, such as furniture, lighting, internal plumbing, fixtures, etc. can be preinstalled in the structural units at the manufacturing site. Thus, in some such examples, assembly of structural units at the job site requires placement of the structural units relative to each other and connection of utilities (e.g., electrical and water) to the structural units. In some examples, the required medical equipment and most or all other equipment can be installed at the job site.

The foregoing and other objects, features, and advantages of the invention will become more apparent from the following detailed description, which proceeds with reference to the accompanying figures.

### BRIEF DESCRIPTION OF THE DRAWINGS

FIG. 1 is a plan view of a building structure comprising a plurality of structural units, according to one example.

FIG. 2 is an enlarged plan view of a first structural unit of the building structure of FIG. 1, according to one example.

FIG. 3 is enlarged plan view of a second structural unit of the building structure of FIG. 1, according to one example.

FIG. 4 is enlarged plan view of a third structural unit of the building structure of FIG. 1, according to one example.

FIG. 5 is enlarged plan view of a fourth structural unit of the building structure of FIG. 1, according to one example.

FIG. 6 is enlarged plan view of a fifth structural unit of the building structure of FIG. 1, according to one example.

FIG. 7 is enlarged plan view of a sixth structural unit of the building structure of FIG. 1, according to one example.

FIG. 8 is enlarged plan view of a seventh structural unit of the building structure of FIG. 1, according to one example.

FIG. 9 is a floor plan of the building structure of FIG. 1, with equipment removed for purposes of illustration.

FIG. 10 is a perspective view of the building structure of FIG. 1.

FIG. 11 is a plan view of a building structure comprising a plurality of structural units, according to another example.

### DETAILED DESCRIPTION

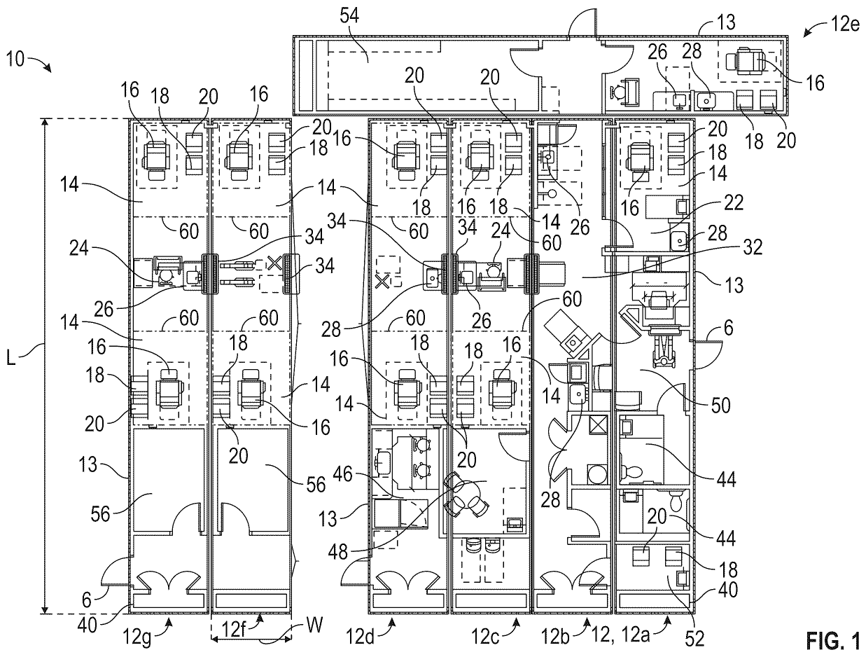

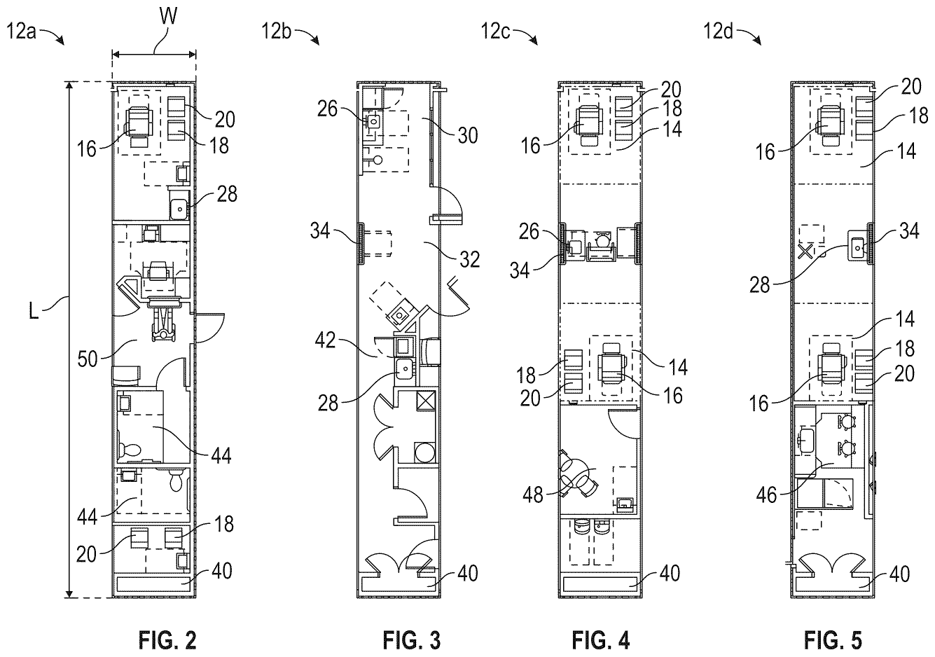

Referring first to FIG. 1, there is shown building structure 10, according to one example, that is configured to function as a medical facility for treating dialysis patients. The building structure 10 comprises an assembly of structural units 12, including a first structural unit 12a, a second structural unit 12b, a third structural unit 12c, a fourth structural unit 12d, a fifth structural unit 12e, a sixth structural unit 12f, and a seventh structural unit 12g. FIGS. 2-8 are enlarged views of the individual structural units 12a-g. As further described below, one or more of the structural units 12a-g can be optional, in some examples. If needed to treat a larger number of patients, additional structural units 12 can be added to the assembly. In some examples, the building structure 10 can be referred to as a modular building structure in that it is comprised of a plurality of similarly or identically sized modules in the form of structural units 12.

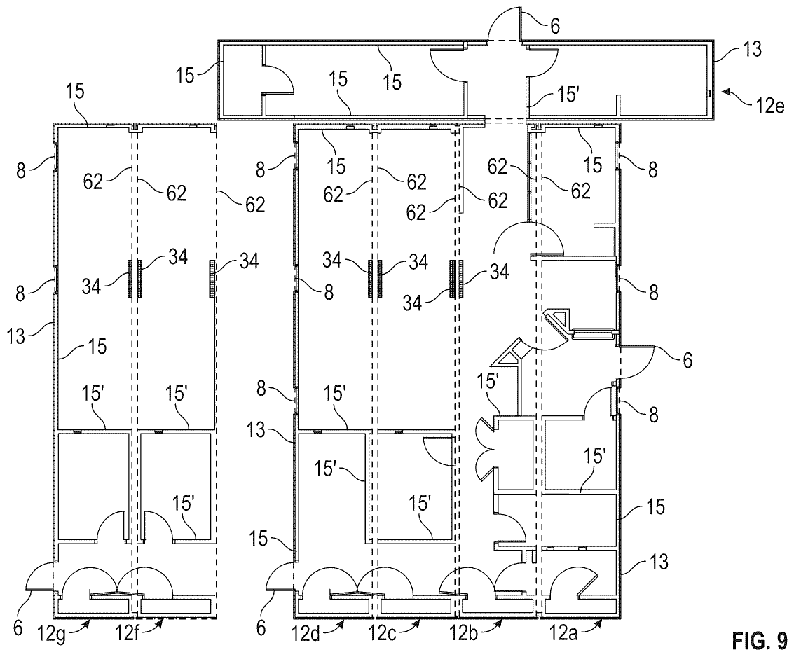

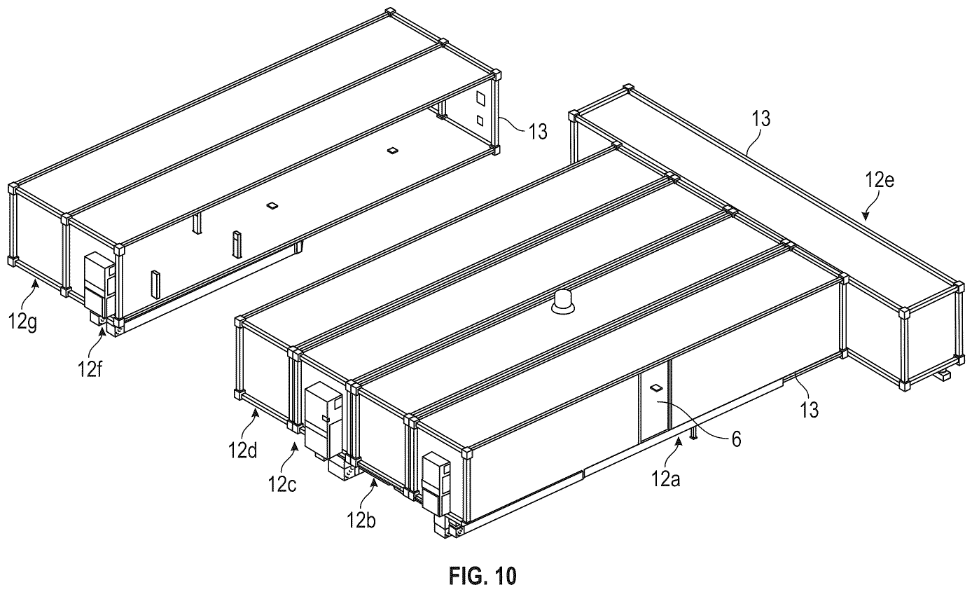

The structural units 12 can comprise shipping containers. In some examples, each of the structural units 12 comprises a standard shipping container having a length L of 53 feet, a width W of 8 feet, six inches (102 inches) and a height of 9 feet. However, the dimensions of the structural units and other dimensions disclosed in the present application can be modified as needed for a particular application. When using standard shipping containers, one or more of the structural units 12 can be modified as needed, such as installing doors 6 and/or windows 8, and forming openings in the outer walls 13 of the containers to provide walking access from one container to an adjacent container when they are assembled together at a job site. For example, as depicted in FIGS. 9-10, one or more structural units 12 can be modified such that at least portions of their outer walls 13 are removed. Accordingly, two or more adjacent structural units 12 can form a continuous interior space or room having a width that is larger than the width W of a single container. Outer walls 13 or portions thereof that have been removed to create larger interior spaces are denoted in FIG. 9 with regular dashed lines 62.

In some examples, as shown in FIG. 9, interior walls 15, such as dry wall panels, can be positioned adjacent to and in facing relationship with the outer walls 13 on the inside of the structural units 12. These interior walls 15 may provide thermal/sound insulation as well as moisture resistance to the outer walls 13. Additional inner walls 15′ may be built within one or more structural units 12 to form separate spaces or rooms that can be used for a variety of purposes, as will be described further below.

Referring again to FIG. 1, the structural units 12a, 12b 12c, 12d, 12f, 12g can be placed side-by-side. In some examples, the structural unit 12e can placed in a perpendicular arrangement relative to the other structural units. Adjacent units can be connected to each other with connectors, which can be in the form of or include any combination of flanges, bolts, screws, clips, fasteners, etc.

One or more of the structural units can have one or more patient stations 14 that include a patient chair 16, a dialysis machine 18, and a water source 20. In the illustrated example, each of the structural units 12a, 12c, 12d, 12f, and 12g includes at least one patient station 14, with structural unit 12a including one patient station 14, and each of structural units 12c, 12d, 12f, and 12g including two patient stations 14. The water source 20 can be a reverse osmosis machine (if the dialysis machine 18 requires purified water) or a fixture that provides tap water (if untreated water can be used with the dialysis machine 18). The dialysis machines 18 can be any type of known dialysis machines, including a hemodialysis, such as an in-center hemodialysis machine, a home hemodialysis machine, or a peritoneal dialysis machine. In some examples, all of the dialysis machines 18 in units 12a, 12c, 12d, 12f, and 12g are in-center hemodialysis machines. If peritoneal dialysis machines are used, then a water source 20 is not needed.

The structural unit 12a includes an isolation room 22, which includes a respective patient station 14. The isolation room 22 is configured to be used for performing dialysis on patients with bloodborne diseases, such as hepatitis.

The structural unit 12c includes a nurse station or clinical worker area (CWA) 24 situated adjacent two patient stations 14 in the structural unit 12c and two patient stations 14 in the structural unit 12d. In some examples, the patient stations 14 in the structural units 12c, 12d can be separated from the CWA 24 by transparent partitions (e.g., glass) such that the CWA 24 is in visual communication with the patient stations 14; that is, a clinical worker seated or standing in the CWA 24 can have visual access of the patients in the chairs 16 of the patient stations 14 in the structural units 12c, 12d. Additionally or alternatively, the structural units 12c, 12d can be free of walls or barriers between the CWA 24 and the patient stations 14 providing visual access therebetween. This allows the clinical worker, such as a nurse or a patient care technician, to be able to visibly monitor four patients undergoing dialysis in those patient stations 14. The dash-dot-dash lines 60 in FIG. 1 represent boundaries of the patient stations 14. Transparent partitions can be placed along boundaries of the patient stations 14 (e.g., along portions of the dash-dot-dash lines 60) in some examples. In some examples, the lines 60 are non-physical boundaries (i.e., there are no walls or partitions along lines 60). At least a portion of the internal wall of the isolation room 22 desirably includes a transparent section (e.g., glass) that allows the clinical worker to monitor a patient in the isolation room from the CWA 24.

Each patient station 14 typically can serve three patients per day. Each dialysis patient typically requires treatment three time per week (e.g., on Monday, Wednesday, and Friday or on Tuesday, Thursday, and Saturday). Thus, each patient station 14 can be used by a total of six patients (assuming a normal workday). Collectively, the patient stations 14 in structural units 12c and 12d can be used by a total of 24 patients, in addition to any patients using the patient station 14 in the isolation room. So long as the medical facility is treating any patients with a bloodborne disease (HIV excepted), regulations require that the isolation room 22 to be used only by such patients.

As noted above, one or more of the units 12 can be optional. If the capacity of the patient stations 14 in units 12c, 12d can adequately serve the geographic area in which the medical facility 10 is located, the units 12f and 12g can be optional. If additional capacity is needed, the units 12f and 12g can be added, which can be used to treat an additional 24 patients. It should be noted that in FIG. 1, the units 12f and 12g are spaced apart from the units 12a, 12b, 12c and 12d for purposes of illustration. If the units 12f and 12g are provided, the unit 12f can be placed against the adjacent unit 12d (with no space or gap therebetween).

A plurality of clean sinks 26 and a plurality of dirty sinks 28 are located within one or more of the structural units 12a, 12b, 12c, 12d, 12f and 12g. In some examples, the plurality of clean and dirty sinks 26, 28 may be mounted to wing walls or partial walls 34 extending upward from the floor of the one or more of the structural units 12a, 12b, 12c, 12d, 12f and 12g. The partial walls 34 may extend a partial distance from the floor toward the ceiling and provide structure for plumbing or other fixtures related to the mounting and operation of the sinks 26, 28. Per regulations, the ratio of the number of clean sinks 26 to the number of patient stations 14 in a patient treatment area desirably is at least 1:4, and the ratio of the number of dirty sinks 28 to the number of patient stations 14 in a patient treatment area desirably is at least 1:8. The clean sinks 26 and the dirty sinks 28 are strategically located to satisfy these requirements, and to accommodate expansion. In particular, in the illustrated example, a clean sink 26 is located in the unit 12c and a dirty sink 28 is located in the unit 12d (although the locations of the sinks 26, 28 can be reversed). A patient treatment area is collectively defined by the patient stations 14 in the units 12c and 12d. If the medical facility 10 does not include additional units 12f and 12g, the placement of at least one clean sink 26 and at least one dirty sink 28 in the units 12c and 12d will satisfy the ratio requirements.

At least one additional clean sink 26 is located in at least one of the additional units 12f and 12g (e.g., in the unit 12g, as shown). Thus, if the additional units 12f and 12g are initially included or added later to the overall structure 10, the presence of the additional clean sink 26 will satisfy the ratio requirements. The dirty sink 28 located in the patient treatment defined by the units 12c and 12d can also be used for the patient treatment area defined by the units 12f and 12g and satisfy the ratio requirements. Thus, in some examples, an additional dirty sink 28 is not required in the additional units 12f and 12g.

If further expansion is required to accommodate more patients, two additional units identical to the units 12c and 12d can be installed side-by-side against the unit 12g (to the left of the unit 12g in the drawings). The two additional units 12c and 12d provide an additional clean sink 26 and an additional dirty sink 28. If still additional capacity is required, two additional units identical to the units 12f and 12g can be installed side-by-side against the previously installed units 12c and 12d. The process of adding an additional pair of units 12c, 12d followed by an additional pair of units 12f, 12g can be repeated as needed to accommodate more patients.

The unit 12b can house various items and/or can be configured for certain uses to satisfy local and/or federal regulations. For example, as best shown in FIG. 3, in some examples, the unit 12b can include a medication prep area 30, a patient prep area 32, and a laboratory 42, which can include a refrigerator, a centrifuge, a sink, etc.

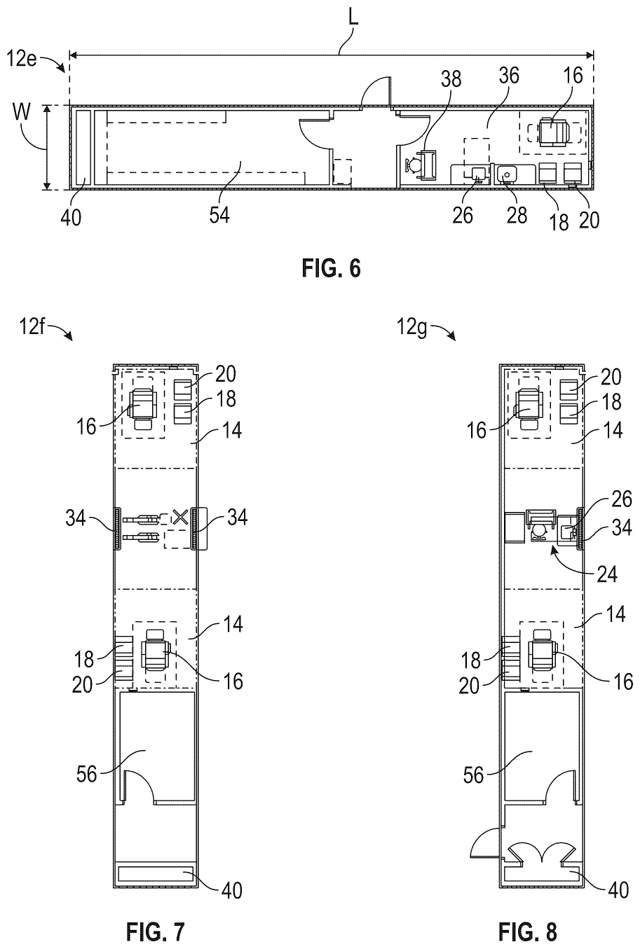

The unit 12e can be configured as a home training unit that provides space for training patients on the use of a home dialysis machine, such as a peritoneal dialysis or a home hemodialysis machine and for examining patients. To such ends, as best shown in FIG. 6, the unit 12e can include a patient station or training station 36 that includes a patient chair 16, a dialysis machine 18 (e.g., peritoneal dialysis or a home hemodialysis machine), a water source 20 (if needed for operating the machine 18), a clean sink 26, and a dirty sink 28. A water source 20 is not needed for operating a peritoneal dialysis machine. A workstation 38 for a clinician, which can include, for example, a desk and a chair, can be located within the patient station 36. The patient station 36 can be an enclosed room within the unit 12e for patient privacy.

In some examples, the units 12a, 12b, 12c, 12d, and 12e, and optionally 12f and 12g, together form a medical facility for treating dialysis patients and which is fully compliant with federal regulations and local codes. The medical facility can be expanded as desired to accommodate more patients by installing additional units, as discussed above.

In some examples, each or one or more of units 12a-g can include a room or closet 40 at one end thereof for housing mechanical and/or electrical equipment, including, but not limited to, HVAC equipment, an electrical panel, servers and other equipment for storing data, etc.

In addition to the patient stations 14 described above, the structural units 12a, 12b, 12c, 12d, 12e, 12f, and 12g may be configured with spaces or rooms arranged to facilitate day-to-day operations. For example, one or more of the units 12a, 12b, 12c, 12d, 12e, 12f, and 12g can include a toilet room 44, a lounge 46, a consultation space 48, and/or a waiting area 50.

In some examples, one or more of the units 12a, 12b, 12c, 12d, 12e, 12f, and 12g can include a repair room 52 where equipment, such as dialysis machines 18, may be serviced. In the example shown in FIG. 1, a repair room 52 is situated in the structural unit 12a. The repair room 52 may comprise a work counter, a supply box which provides tap water, a clean sink for hand washing, and/or storage cabinets.

The structural units 12a, 12b, 12c, 12d, 12e, 12f, and 12g may also comprise storage areas 54 and/or flex spaces 56 that can be readily reconfigured to support different functions, workflows, or occupancy needs. As shown in FIG. 1, the structural units 12f, 12g each comprise such a flex space 56 and the structural unit 12e is configured with a storage area 54. Additional details regarding these spaces can be found in Example A below. The structural units 12a, 12b, 12c, 12d, 12e, 12f, and 12g may be configured with any combination of such rooms or spaces to support operational requirements of the medical facility.

As further shown in the figures, one or more of the structural units can be provided with exterior doors 6 to permit access to the building structure 10. In the illustrated example, units 12a, 12d, 12g, and 12e have exterior doors 6.

In some examples, the structure 10 can be disassembled into the individual units 12, which can then be transported to another location in need of such a medical facility. For example, if the structure 10 is initially installed in a rural town or city in which a hospital is constructed or a conventional brick and mortar dialysis center is opened, the structure 10 can be disassembled and the units 12 can be transported to a new location and re-assembled. In some examples, any of units 12a, 12c, 12d, 12g and 12f can be used alone or in combination with each other for treating dialysis patients during a state of emergency. For example, even if they are part of an existing medical facility currently in use, one or more of units 12a, 12c, 12d, 12g and 12f can be disconnected from adjacent units and shipped, such as via tractor trailer, to a location experiencing a natural disaster or other event declared a state of emergency.

Additional details regarding the structure 10 in accordance with some examples are described below in Example A.

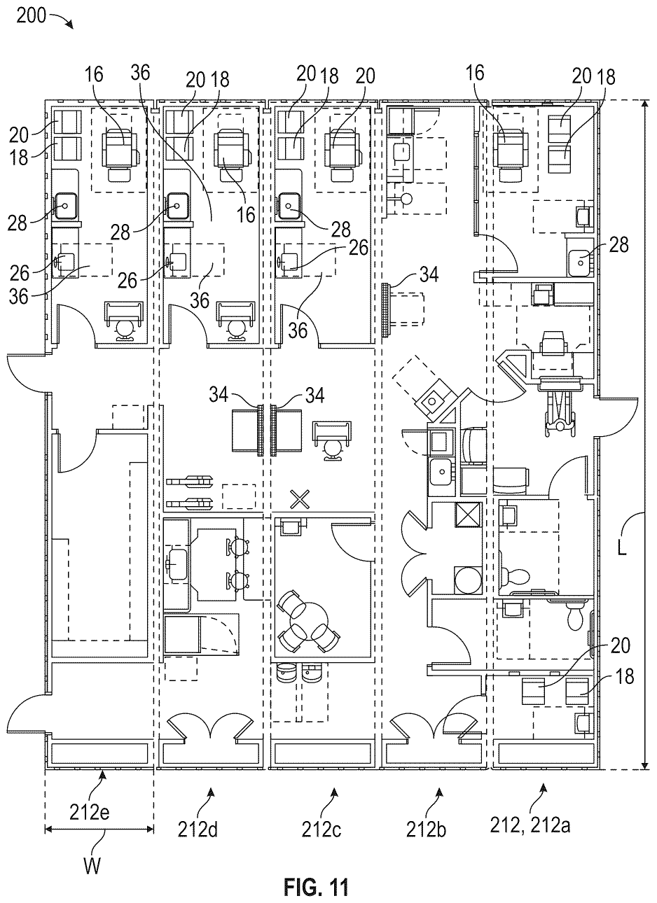

Referring now to FIG. 11, is there is shown modular building structure 200, according to one example, that is configured to function as a medical facility for training patients on home dialysis. The structure 200 can comprise a plurality of structural units 212, including a first structural unit 212a, a second structural unit 212b, a third structural unit 212c, a fourth structural unit 212d, and a fifth structural unit 212e. The structural units 212a-e can be positioned side-by-side relative to each other as shown.

In some examples, the unit 212a can be identical to the unit 12a shown in FIG. 1, and the unit 212b can be identical to the unit 12b in FIG. 1. In some examples, the unit 212e can be identical to the unit 12e in FIG. 1 and can include a patient station 36 for training a patient on home dialysis use. The units 212c and 212d are similar to the unit 212e and each include a patient station 36 for training a patient on home dialysis use.

Additional details regarding the structure 200 in accordance with some examples are described below in Example B.

### Example A: Additional Details for Constructing the Structure 10 According to One Example

Below is a functional program narrative as it applies to the 2018 Facility Guidelines Institute Guidelines for Design and Construction of Outpatient Facilities, particularly Chapter 2.10 for Renal Dialysis Centers and how it relates directly to an Outpatient Chronic Facility.

Facility Size: The square footage can vary based on functional program.

Functional Program: The facility can be a specialty medical center with a singular focus of providing outpatient chronic dialysis treatments, training and the ancillary activities related to the provision of these treatments and trainings. It need not be attached to a hospital or other medical clinic/center nor provide acute dialysis care.

Site: The site of the proposed outpatient dialysis center can be determined by the needs of a community. There is adequate parking for both staff and patients at a ratio determined by local jurisdiction. Handicapped parking spaces as well as the appropriate accessible access route to the clinic's main entrance can follow local jurisdiction regulations. The facility need not provide transportation for patients. The patients from the surrounding community can rely on their own means of transportation through family, friends, medical, or public transportation. The site can provide truck access for the delivery of supplies on a weekly basis or as designated by the Facility Administrator.

Shell: The shell can be a metal building.

Accommodations for Care of Patients of Size: The facility's patient areas have provisions to provide treatment for patients of size such as doors 3′-6″ in width to accommodate larger and electric wheelchairs, a Hoyer lift kept on site, bariatric wheelchair, and a bariatric patient treatment chair.

Patient Care and Diagnostic Areas may include the following Examination room(s): An exam room if required by state or local regulations can provide a minimum of 100 square feet of private area for the examination of patients and include a clean sink of hand washing and a work counter if required by local jurisdiction regulations.

In-Center Hemo Dialysis Treatment Area(s): The treatment floor is dedicated as a treatment area and can remain separate from public and administrative areas.

Number of Patient Stations: The outpatient dialysis center can provide in-center hemodialysis treatments. Each patient station 14 includes a dialysis chair 16, dialysis machine 18, and water source 20. The water source 20 may be either a portable reverse osmosis machine (RO) or water box that supplies either tap or RO water. The ultimate operations and staffing can be two (2) and/or three (3) treatment shifts per day, Monday through Saturday. Normally this will break down into three (3) shifts on Monday, Wednesday, and Friday; and two (2) shifts on Tuesday, Thursday, and Saturday.

Space Requirements: This center provides chairs for the patient's use during treatment. Therefore the open treatment floor provides a varied number of patient stations 14 which provides a minimum of 80 square feet per station 14 or as required by local jurisdiction regulations. The patient stations are arranged to provide the required minimum 4′-0″ clearance between station chairs 16, 3′-0″ clearance between the chair 16 and adjacent walls, and at least 2′-0″ at the end of the chair 16 before leaving the designated patient area. The transport aisleways for use by a stretcher are a minimum of 44 inches or as designated by local jurisdiction regulations.

Patient Privacy: The outpatient dialysis center can provide temporary movable screens when applicable to allow patient privacy in the center.

Hand-washing Stations: The hand washing stations are distributed throughout the treatment floor to provide a hygienic environment. The arrangement of clean sinks 26 on the treatment floor is at a minimum ratio of 1:4 with the patient stations 14. The number of dirty sinks 28 on the treatment floor is at a minimum ratio of 1:8 with the patient stations 14. The patient preparation area includes a hand washing station close to the treatment floor entry.

P.D/H.T. Room: The Home Training (HHD) and/or Peritoneal Dialysis (PD) are 150 square feet or as defined by local jurisdiction regulations. It provides a private training/treatment area for both peritoneal and home hemo dialysis modalities, a dirty sink 28, a clean sink 26 for hand washing, a work counter, and a home dialysis machine connection box. The connection box provides both a domestic water supply and separate drain for fluid disposal similar to those readily found in patient homes.

Special Treatment Area(s) may include the following. Isolation Room(s): The patients are routinely tested for airborne infections. Those patients presenting with airborne infections can be transferred to another clinic or facility which is able to meet their special treatment needs. Each facility has a room designed for the treatment of patients with a bloodborne pathogen in accordance with the CMS Federal conditions regarding isolation. The patient isolation station 22 can be a private treatment station designated for the treatment of patients with bloodborne infections in accordance with the Centers for Medicare and Medicaid Services Guidelines. The room is a minimum of 120 square feet. The room provides a single patient station 14, a clean sink 26, and minimum 7′-0″ high partitions with a self-closing door to prevent the spread of blood splatter. These partitions may be constructed of aluminum and glass to provide a clear sight line to the isolated patient.

Airborne isolation rooms are not required for outpatient dialysis facilities per the sections below:

Referencing V tag 128 IGs: Since Hepatitis B is not airborne, the walls of the room do not need to reach the ceiling but would need to go to the floor in order to contain blood spills and the door must be closed during times when blood spurting or spattering is possible, e.g., at initiation and termination of treatment. The walls need to allow visual monitoring of the patients in the room (unless a staff member is continually present in the room) and to contain any potential blood or fluid spills or spurts.

Referencing V128 CMS FAQs/Centers for Medicare & Medicaid Services Version 1.3: In an outpatient dialysis facility, an isolation room 22 is required for isolation of patients who are Hepatitis B+, which is a bloodborne virus. It is not expected that outpatient dialysis facilities would have the capacity to isolate airborne organisms.

Support Areas for the Renal Dialysis Treatment Center may include the following. Nurse Station(s): The Nurse Stations (e.g., nurse stations or CWA 24) are located in an area to provide visual observation of the patients at a ratio of approximately one knee space for every four patient stations 14.

Medication Safety Zone: The Medication Preparation area (Med Prep.) 30 is considered a clean area and provides a clean sink 26 in close proximity. The location provides countertop space, locking storage cabinets and locking refrigerator. The square footage is 48 square feet. This may vary depending on location.

Nourishment Station: N/A unless otherwise required by local jurisdiction.

Clean Workroom or Clean Supply Room: N/A unless otherwise required by local jurisdiction.

Soiled Workroom or Soiled Holding Room: N/A unless otherwise required by local jurisdiction.

Equipment and Supply Storage: N/A unless otherwise required by local jurisdiction.

Clean Linen Storage: N/A unless otherwise required by local jurisdiction.

Clinical equipment and supply storage: N/A unless otherwise required by local jurisdiction.

Wheelchairs: A storage area is provided on the treatment floor for wheelchairs storage at a minimum ratio of one wheelchair per four patient stations.

Support Areas for Staff may include the following. The staff support area includes a lounge room with a square footage of 172. The lounge includes lockers, a refrigerator, along with table and chairs. Other staff support areas include a conference room with square footage of 192 square feet. Teammate toilet rooms 44 are 70 square feet. Offices are available as needed and may be 92 square feet or 156 square feet.

Support Areas for Patients may include the following. The support areas at this facility include toilet rooms which are 54 square feet, equipped with emergency nurse calls, and storage of personal items will be via coat rack at the patient prep area 32 (P.P.). These areas are designed according to the currently adopted handicap accessibility code.

Dialysis Support Facilities may include the following. Dialyzer Reprocessing: N/A unless otherwise required by local jurisdiction.

Dialysate Preparation Area: N/A unless otherwise required by local jurisdiction.

Equipment Repair Room: The equipment repair room 52, encompassing 144 square feet, provides space for the local Biomed administrator to perform repair and maintenance to the equipment used during the dialysis process. This area provides a work counter, a supply box which provides either tap or RO water and concentrate similar to each patient station, a clean sink 26 for hand washing, and storage cabinets.

Eyewash Station: Emergency eye wash stations are provided in the following areas of the clinic: at a nurse station 24 on the treatment floor, and within the equipment repair room 52 and the RO room if there is a RO system installed. The fixture within the equipment repair room 52 is hose mounted to allow emergency drench shower function if needed.

Building Support Facilities may include the following. Waste Management: The existing or new dumpster location and predetermined collection times can be coordinated with the local sanitation department. The collection of infectious waste can be coordinated by the Facility Administrator to be picked up by a company licensed in the disposal of infectious medical waste. Waste collection can be located in a room which measures 40 square feet.

Environmental Services: The environmental services room is 36 square feet. The room will provide space for cleaning supply storage, a hand washing sink OR hand sanitizer station, and a mop sink.

Public Areas may include the following. The public areas at this clinic include a waiting room 50 (which is 100 square feet), and toilet room 44 (which is 56 square feet). The waiting room 50 provides space for both patients and visitors to wait comfortably for extended periods of time if necessary. There can be a water fountain that will be located within the facility. There can be a public telephone jack. This area is accessible in compliance with the currently adopted handicap accessibility code.

Administrative Areas: The administrative areas include clerical spaces such as the reception area (which is 85 square feet). Each nurse station 24 located on the treatment floor provides clerical space to be used for charting and monitoring purposes.

Medical Records: N/A unless otherwise required by local jurisdiction. Medical records are electronic.

Surfaces: The materials used in the facility have been selected to be easy to clean and disinfect such as fiber reinforced wall protection, solid surface, and plastic laminate.

Building Systems may include the following. Heating, Ventilation, and Air Conditioning (HVAC) Systems. This renal dialysis facility complies with all project pertinent requirements listed in ANSI/ASHRAE/ASHE Standard 170: Ventilation of Health Care Facilities.

Electrical: All electrical material and equipment serving this dialysis facility complies with all applicable sections of NFPA 70, NFPA 99 and other required standard.

Electrical Distribution and Transmission: Electrical equipment are complied with NFPA 70 and NFPA 99 requirements, accessible to authorized person and in dry, ventilated spaces. All receptacles serving treatment area are hospital grade and GFCI protected.

Lighting: Fixed recessed LED lighting are provided in the exam and treatment rooms. Egress lighting can be powered via central inverter and bypass relays.

Electrical Equipment: All electrical wiring and devices serving the treatment room and exam to be hospital grade and complied with NFPA 99 and NFPA 70.

Plumbing: The piping used to distribute the high purity water used in dialysis, the dialysate concentrate(s), and/or bicarb solution is distributed with either a chase or a T-Box solution. The indirect drain system will dump spent dialysate fluid into the sanitary system. The indirect connections will meet the required air gap codes to prevent backflow.

Plumbing: The process piping can be installed separate from the domestic water supply and is sanitary.

Water Treatment Equipment Room: N/A unless otherwise needed.

Communication Systems may include the following. Call System: Nurse call stations comply with requirement for outpatient facilities.

Telecommunications and information: A dedicated IT room can be provided to terminate telecommunication service.

Fire Alarm Systems: Fire alarm system can be provided in accordance with NFPA 101 and NFPA 72.

### Example B: Additional Details for Constructing the Structure 200, According to One Example

Below is a functional program narrative as it applies to the 2018 Facility Guidelines Institute Guidelines for Design and Construction of Outpatient Facilities, particularly Chapter 2.10 for Renal Dialysis Centers and how it relates directly to an Outpatient Chronic Facility.

Facility Size: The square footage can vary based on functional program.

Functional Program: The facility can be a specialty medical center with a singular focus of providing outpatient chronic dialysis treatments, training and the ancillary activities related to the provision of these treatments and trainings. It need not be attached to a hospital or other medical clinic/center nor provide acute dialysis care.

Site: The site of the proposed outpatient dialysis center can be determined by the needs of a community. There is adequate parking for both staff and patients at a ratio determined by local jurisdiction. Handicapped parking spaces as well as the appropriate accessible access route to the clinic's main entrance can follow local jurisdiction regulations. The facility need not provide transportation for patients. The patients from the surrounding community can rely on their own means of transportation through family, friends, medical, or public transportation. The site provides truck access for the delivery of supplies on a weekly basis or as designated by the Facility Administrator.

Accommodations for Care of Patients of Size: The facility's patient areas have provisions to provide treatment for patients of size such as doors 3′-6″ in width to accommodate larger and electric wheelchairs, a Hoyer lift kept on site, bariatric wheelchair, and bariatric patient treatment chair.

Patient Care and Diagnostic Areas may include the following. Examination Room(s): An exam room can provide a minimum of 100 square feet of private area for the examination of patients and include a clean sink 26 of hand washing and a work counter if required by local jurisdiction regulations.
In-Center Hemo Dialysis Treatment Area(s)—N/A
Number of Patient Stations: N/A
Space Requirements: N/A
Patient Privacy: N/A
Hand-washing Stations: N/A

P.D/H.T. Room: The Home Training (HHD) and/or Peritoneal Dialysis (PD) are 120 square feet or as defined by local jurisdiction regulations. It provides a private training/treatment area for both peritoneal and home hemo dialysis modalities, a dirty sink 28, a clean sink 26 for hand washing, a work counter, and a home dialysis machine connection box. The connection box provides both a domestic water supply and separate drain for fluid disposal similar to those readily found in patient homes.

Special Treatment Area(s) may include the following. Isolation Room(s): The patients are routinely tested for airborne infections. Those patients presenting with airborne infections can be transferred to another clinic or facility which is able to meet their special treatment needs. Each facility has a room designed for the treatment of patients with a bloodborne pathogen in accordance with the CMS Federal conditions regarding isolation. The patient isolation station 22 will be a private treatment station designated for the treatment of patients with bloodborne infections in accordance with the Centers for Medicare and Medicaid Services Guidelines. The room is a minimum of 120 square feet. The room provides a single patient station 14, a clean sink 26, and minimum 7′-0″ high partitions with a self-closing door to prevent the spread of blood splatter. These partitions may be constructed of aluminum and glass to provide a clear sight line to the isolated patient.

Airborne isolation rooms are not required for outpatient dialysis facilities per the sections below:

Referencing V tag 128 IGs: Since Hepatitis B is not airborne, the walls of the room do not need to reach the ceiling but would need to go to the floor in order to contain blood spills and the door must be closed during times when blood spurting or spattering is possible, e.g., at initiation and termination of treatment. The walls need to allow visual monitoring of the patients in the room (unless a staff member is continually present in the room) and to contain any potential blood or fluid spills or spurts.

Referencing V128 CMS FAQs/Centers for Medicare & Medicaid Services Version 1.3: In an outpatient dialysis facility, an isolation room is required for isolation of patients who are Hepatitis B+, which is a bloodborne virus. It is not expected that outpatient dialysis facilities would have the capacity to isolate airborne organisms.

Support Areas for the Renal Dialysis Treatment Center may include the following. Nurse Station(s): The Nurse Stations (e.g., nurse stations 24) can be located in an area to provide visual observation of the patients at a ratio of approximately one knee space for every four patient stations 14.

Medication Safety Zone: The Medication Preparation area (Med Prep.) is considered a clean area and provides a clean sink 26 in close proximity. The location provides countertop space, locking storage cabinets and locking refrigerator. The square footage is 48 square feet. This may vary depending on location.

Nourishment Station: N/A unless otherwise required by local jurisdiction.

Clean Workroom or Clean Supply Room: N/A unless otherwise required by local jurisdiction.

Soiled Workroom or Soiled Holding Room: N/A unless otherwise required by local jurisdiction.

Equipment and Supply Storage: N/A unless otherwise required by local jurisdiction.

Clean Linen Storage: N/A unless otherwise required by local jurisdiction.

Clinical equipment and supply storage: N/A unless otherwise required by local jurisdiction.

Wheelchairs: A storage area is provided for wheelchairs storage at a minimum ratio of one wheelchair per four patient stations.

Support Areas for Staff may include the following. The Staff support area includes a lounge. The lounge includes lockers, refrigerator along with table and chairs. Other staff support areas include a conference room, with square footage of 83 square feet. Teammate toilet rooms have a square footage of 61 square feet.

Support Areas for Patients may include the following. The support areas at this facility include toilet rooms (56 square feet), equipped with emergency nurse calls, and storage of personal items will be via coat rack at patient prep area 32 (P.P.) which is 50 square feet. These areas are designed according to the currently adopted handicap accessibility code.

Dialysis Support Facilities may include the following. Dialyzer Reprocessing: N/A unless otherwise required by local jurisdiction.

Dialysate Preparation Area: N/A unless otherwise required by local jurisdiction.

Equipment Repair Room: The equipment repair room, encompassing 45 square feet, provides space for the local Biomed administrator to perform repair and maintenance to the equipment used during the dialysis process. This area provides a work counter, a supply box which provides either tap water, a clean sink 26 for hand washing, and storage cabinets.

Eyewash Station: Emergency eye wash stations are provided in the following areas of the clinic: at a nurse station (e.g., CWA 24) on the treatment floor, and within the equipment repair room 52. The fixture within the equipment repair room is a hose mounted to allow emergency drench shower function if needed.

Building Support Facilities may include the following. Waste Management: The existing or new dumpster location and predetermined collection times can be coordinated with the local sanitation department. The collection of infectious waste can be coordinated by the Facility Administrator to be picked up by a company licensed in the disposal of infectious medical waste.

Environmental Services: The environmental services room is 20 square feet. The room can provide space for cleaning supply storage, a hand washing sink or hand sanitizer station, and a mop sink.

Public Areas may include the following. The public areas at this clinic include a waiting room 50 (which is 100 square feet), and toilet room (which is 56 square feet). The waiting room provides space for both patients and visitors to wait comfortably for extended periods of time if necessary. There can be a water fountain that will be located within the facility. There can be a public telephone jack. This area is accessible in compliance with the currently adopted handicap accessibility code.

Administrative Areas: The administrative areas include clerical spaces such as the reception area (which is 48 square feet). Each training room provides clerical space to be used for charting and monitoring purposes.

Medical Records: N/A unless otherwise required by local jurisdiction. Medical records are electronic.

Surfaces: The materials used in the facility have been selected to be easy to clean and disinfect such as fiber reinforced wall protection, solid surface, and plastic laminate.

Building Systems may include the following. Heating, Ventilation, and Air Conditioning (HVAC) Systems: This renal dialysis facility complies with all project pertinent requirements listed in ANSI/ASHRAE/ASHE Standard 170: Ventilation of Health Care Facilities.

Electrical: All electrical material and equipment serving this dialysis facility complies with all applicable sections of NFPA 70, NFPA 99 and other required standard.

Electrical Distribution and Transmission: Electrical equipment are complied with NFPA 70 and NFPA 99 requirements, accessible to authorized person & in dry, ventilated spaces. All receptacles serving treatment area are hospital grade and GFCI protected.

Lighting: Fixed recessed LED lighting are provided in Exam and treatment room. Egress lighting to be powered via central inverter and bypass relays.

Electrical Equipment—All electrical wiring and devices serving treatment room and exam to be hospital grade and complied with NFPA 99 and NFPA 70.

Plumbing-Piping: Installed as applicable to local code requirements.

Plumbing: Process piping is not applicable in a Home Training Clinic

Water Treatment Equipment Room: N/A unless otherwise needed

Communication Systems may include the following. Call System—Nurse call stations are complied with requirement for outpatient facilities.

Telecommunications and information: A dedicated IT room is provided to terminate telecommunication service.

Fire Alarm Systems: Fire alarm system will be provided in accordance with NFPA 101 and NFPA 72.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

### Example 1

A structure for use as a medical facility, the structure comprising: a plurality of discrete structural units placed side-by-side relative to each other, wherein at least one structural unit of the plurality of discrete structural units has at least one patient station that includes a patient chair and a dialysis machine.

### Example 2

The structure of any example herein, particularly example 1, wherein the plurality of discrete structural units comprises a first structural unit and a second structural unit positioned adjacent the first structural unit, wherein the first and second structural units each comprise at least one patient station.

### Example 3

The structure of any example herein, particularly example 2, wherein one of the first and second structural units comprises a clean sink and the other of the first and second structural units comprises a dirty sink.

### Example 4

The structure of any example herein, particularly example 3, wherein the first and second structural units each comprise two patient stations.

### Example 5

The structure of any example herein, particularly example 4, wherein a clinical worker area is arranged within one of the first and second structural units and is in visual communication with the two patient stations of both the first and second structural units.

### Example 6

The structure of any example herein, particularly example 1, wherein each of the plurality of discrete structural units is a shipping container.

### Example 7

The structure of any example herein, particularly example 3, wherein the plurality of discrete structural units comprises a third structural unit positioned adjacent the first or second structural units and a fourth structural unit positioned adjacent the first, second, or third structural units, wherein the third and fourth structural units each comprise one or more patient stations and one of the third and fourth structural units comprises a clean sink.

### Example 8

The structure of any example herein, particularly example 7, wherein the third and fourth structural units are free of a dirty sink.

### Example 9

The structure of any example herein, particularly example 7, wherein the plurality of discrete structural units comprises a fifth structural unit positioned adjacent any of the first, second, third, or fourth structural units, wherein the fifth structural unit comprises a training station and at least one patient station.

### Example 10

The structure of any example herein, particularly example 1, wherein for every four patient stations of the at least one patient station, the plurality of discrete structural units comprises no more than one clean sink.

### Example 11

The structure of any example herein, particularly example 1, wherein for every eight patient stations of the at least one patient station, the plurality of discrete structural units comprises no more than one dirty sink.

### Example 12

The structure of any example herein, particularly example 1, wherein any one of the plurality of discrete structural units comprises a room configured as an isolation room.

### Example 13

The structure of any example herein, particularly example 1, wherein each patient station comprises a water source.

### Example 14

A structure for use as a medical facility, the structure comprising: a shipping container; a patient chair located in the shipping container; and a dialysis machine located in the shipping container.

### Example 15

The structure of any example herein, particularly example 14, wherein the shipping container is connected to at least one additional shipping container comprising a patient chair and a dialysis machine.

### Example 16

The structure of any example herein, particularly example 15, wherein the shipping container comprises a clean sink and the at least one additional shipping container comprises a dirty sink.

### Example 17

The structure of any example herein, particularly example 15, wherein the at least one additional shipping container comprises at least three additional shipping containers.

### Example 18

The structure of any example herein, particularly example 17, wherein for every four patient chairs or four dialysis machines, the structure comprises a single clean sink.

### Example 19

The structure of any example herein, particularly example 17, wherein for every eight patient chairs or eight dialysis machines, the structure comprises a single dirty sink.

### Example 20

A method of forming a structure for use as a medical facility, the method comprising: connecting a first structural unit to a second structural unit disposed adjacent the first structural unit, wherein each of the first and second structural units comprises at least one patient chair and at least one dialysis machine.

### Example 21

The method of any example herein, particularly example 20, further comprising arranging a clinical worker area within one of the first and second structural units such that the at least one patient chair and the at least one dialysis machine in each of the first and second structural units is visible from the clinical worker.

### Example 22

The method of any example herein, particularly example 20, further comprising configuring the first structural unit with a clean sink and the second structural unit with a dirty sink.

### Example 23

The method of any example herein, particularly example 20, further comprising: connecting a third structural unit to the first or second structural units; and connecting a fourth structural unit to the first, second, or third structural units, wherein each of the third and fourth structural units comprises one or more patient chairs and one or more dialysis machines.

### Example 24

The method of any example herein, particularly example 23, further comprising connecting at least a fifth structural unit to any of the first, second, third, or fourth structural units, wherein the fifth structural unit comprises a training station, a medication prep area, a patient prep area, or a laboratory.

### Example 25

The method of any example herein, particularly example 24, further comprising removing any of the first, second, third, fourth, and fifth structural units from the structure.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as my invention all that comes within the scope and spirit of these claims.

1. A structure for use as a medical facility, the structure comprising:
a plurality of discrete structural units placed side-by-side relative to each other,
wherein at least one structural unit of the plurality of discrete structural units has at least one patient station that includes a patient chair and a dialysis machine.

2. The structure of claim 1, wherein the plurality of discrete structural units comprises a first structural unit and a second structural unit positioned adjacent the first structural unit, wherein the first and second structural units each comprise at least one patient station.

3. The structure of claim 2, wherein one of the first and second structural units comprises a clean sink and the other of the first and second structural units comprises a dirty sink.

4. The structure of claim 3, wherein the first and second structural units each comprise two patient stations.

5. The structure of claim 4, wherein a clinical worker area is arranged within one of the first and second structural units and is in visual communication with the two patient stations of both the first and second structural units.

6. The structure of claim 1, wherein each of the plurality of discrete structural units is a shipping container.

7. The structure of claim 3, wherein the plurality of discrete structural units comprises a third structural unit positioned adjacent the first or second structural units and a fourth structural unit positioned adjacent the first, second, or third structural units, wherein the third and fourth structural units each comprise one or more patient stations and one of the third and fourth structural units comprises a clean sink.

8. The structure of claim 7, the third and fourth structural units are free of a dirty sink.

9. The structure of claim 7, wherein the plurality of discrete structural units comprises a fifth structural unit positioned adjacent any of the first, second, third, or fourth structural units, wherein the fifth structural unit comprises a training station and at least one patient station.

10. The structure of claim 1, wherein for every four patient stations of the at least one patient station, the plurality of discrete structural units comprises no more than one clean sink.

11. The structure of claim 1, wherein for every eight patient stations of the at least one patient station, the plurality of discrete structural units comprises no more than one dirty sink.

12. The structure of claim 1, wherein any one of the plurality of discrete structural units comprises a room configured as an isolation room.

13. The structure of claim 1, wherein each patient station comprises a water source.

14. A structure for use as a medical facility, the structure comprising:
a shipping container;
a patient chair located in the shipping container; and
a dialysis machine located in the shipping container.

15. The structure of claim 14, wherein the shipping container is connected to at least one additional shipping container comprising a patient chair and a dialysis machine.

16. The structure of claim 15, wherein the shipping container comprises a clean sink and the at least one additional shipping container comprises a dirty sink.

17. The structure of claim 15, wherein the at least one additional shipping container comprises at least three additional shipping containers.

18. The structure of claim 17, wherein for every four patient chairs or four dialysis machines, the structure comprises a single clean sink.

19. The structure of claim 17, wherein for every eight patient chairs or eight dialysis machines, the structure comprises a single dirty sink.

20. A method of forming a structure for use as a medical facility, the method comprising:
connecting a first structural unit to a second structural unit disposed adjacent the first structural unit, wherein each of the first and second structural units comprises at least one patient chair and at least one dialysis machine.
